(19) United States
(12) Patent Application Publication
(10) Pub. No.: US 2024/0386244 A1
(43) Pub. Date: Nov. 21, 2024

(54) COGNITIVE TRAINING MATERIAL GENERATION METHOD, CONGNITIVE TRAINING METHOD, DIVICE, AND MEDIUM

(71) Applicant: ZHEJIANG LAB, Hangzhou (CN)

(72) Inventors: Yu ZHANG, Hangzhou (CN); Huan ZHANG, Hangzhou (CN); Jing ZHANG, Hangzhou (CN); Yuanyuan LI, Hangzhou (CN); Zhichao WANG, Hangzhou (CN); Tianzi JIANG, Hangzhou (CN)

(21) Appl. No.: 18/427,844

(22) Filed: Jan. 31, 2024

Related U.S. Application Data
(63) Continuation of application No. PCT/CN2023/127496, filed on Oct. 30, 2023.

(30) Foreign Application Priority Data
May 15, 2023 (CN) 202310538580.9

(51) Int. Cl. G06N 3/0455; A61B 5/055; G01R 33/48; G01R 33/56; G06N 3/08

(52) U.S. Cl. CPC G06N 3/0455; A61B 5/055; G01R 33/4806; G01R 33/5608; G06N 3/08

(57) ABSTRACT

A cognitive training material generation method, a cognitive training method, a device, and a medium are provided. The cognitive training material generation method includes: acquiring a first feature and a second feature, the first feature including a multimedia material and semantic information corresponding to the multimedia material, the second feature including a magnetic resonance representation; fitting the first feature and the second feature, obtaining a semantic map according to a fitting result and a preset brain map, and acquiring target semantic information corresponding to a target point according to the semantic map; taking the first feature as input of a deep learning model and the second feature as a constraint of the deep learning model, training the deep learning model, and determining a weight parameter of the deep learning model; generating a cognitive training material according to the target semantic information and the weight parameter of the deep learning model.

### CROSS-REFERENCE TO RELATED APPLICATIONS

This application is a continuation of international patent application No. PCT/CN2023/127496, filed on Oct. 30, 2023, which claims priority to Chinese patent applications No. 202310538580.9, filed on May 15, 2023, titled “COGNITIVE TRAINING MATERIAL GENERATION METHOD, CONGNITIVE TRAINING METHOD, DIVICE, AND MEDIUM”. The contents of the above applications are hereby incorporated by reference.

### TECHNICAL FIELD

The present disclosure generally relates to the field of deep learning, and in particular, to a cognitive training material generation method, a cognitive training method, a device, and a medium.

### BACKGROUND

A human brain responds differently to different things, and magnetic resonance imaging (MRI) is a non-invasive way of detecting brain response with high spatial resolution. A cerebral cortex can be divided into regions based on brain function, such as a temporal lobe region for auditory function and an occipital lobe for visual function, and this division is shared across individuals. When a subject is looking at a graph, in addition to stimulation of an occipital cortex, semantic meaning of the graph also stimulates a medial and lateral parietal cortex, a temporal cortex, and a lateral prefrontal cortex. A mapping between sensory stimuli and brain MRI response signals can be established by deep learning, so that the sensory stimuli can be selected according to cortical target points that need to be responded to. Enhancement of neural activity at corresponding target points by cognitive training could potentially be a way to improve corresponding senses such as attention, memory, and thinking.

Cognitive training is a training methodology designed to enhance human cognitive abilities and improve thinking processes and behavioral performance. This is a systematic cognitive intervention that improves thinking and behavioral performance of people by training and practicing a range of cognitive skills. Cognitive training can be customized to suit different cognitive abilities of an individual in areas such as improving attention, stimulating memory, improving decision making and solving problem. Because cognitive training can improve cognitive abilities of people, it can theoretically be applied to all age groups, including children, adolescents, and adults, and is important in learning, work, and life.

In related techniques, cognitive training has not considered brain science but only from a perspective of application, such as designing a game to avoid Alzheimer disease. In recent years, there have also been studies on stimulating known functional brain regions by transcranial electrical stimulation, but size and effect of electrical stimulation varies from person to person, electrical stimulation cannot stimulate deep brain regions and does not allow for individualized implementation. Therefore, the current cognitive training has not yet established a semantic map of the brain between a cognitive training material and a response of a brain region via MRI to position subsidiarily, and there is no measure of whether the cognitive training material can accurately and efficiently stimulate functional cortical target points in the brain that need to be trained prior to the cognitive training.

For the issue of a problem in the related art of not being able to acquire the cognitive training material that can accurately stimulate functional cortical target points in the brain that need to be trained, no effective solution has been proposed.

### SUMMARY

According to various embodiments of the present disclosure, a cognitive training material generation method, a cognitive training method, a device, and a medium are provided.

In a first aspect, a cognitive training material generation method is provided in the present disclosure, including:
acquiring a first feature and a second feature, the first feature including a multimedia material and semantic information corresponding to the multimedia material, the second feature including a magnetic resonance characterization, and an association relationship existing between the first feature and the second feature;
fitting the first feature and the second feature, obtaining a semantic map according to a fitting result and a preset brain map, and acquiring target semantic information corresponding to a target point according to the semantic map;
taking the first feature as an input of a deep learning model and the second feature as a constraint of the deep learning model, training the deep learning model, and determining a weight parameter of the deep learning model when the deep learning model satisfies a convergence condition; and
generating a cognitive training material according to the target semantic information and the weight parameter of the deep learning model.

In some embodiments, the generating the cognitive training material further includes:
inputting a first cognitive training material generated according to the target semantic information and the weight parameter of the deep learning model into the deep learning model, and predicting a second feature corresponding to the first cognitive training material;
determining whether the second feature corresponding to the first cognitive training material satisfies a preset condition; and
screening the first cognitive training material according to a determining result to obtain a second cognitive training material.

In some embodiments, the acquiring the first feature further includes:
extracting a feature of the multimedia material by a convolutional neural network, encoding the semantic information of the multimedia material, and taking the feature of the multimedia material and encoded semantic information as the first feature.

In some embodiments, the acquiring the second feature further includes:
acquiring a functional magnetic resonance signal corresponding to the first feature, and extracting a feature of the functional magnetic resonance signal, and obtaining a signal feature; and
mapping the signal feature to a cerebral cortex, and taking a mapped signal feature as the second feature.

In some embodiments, after taking the mapped signal feature as the second feature, the method further includes:
acquiring a structural-state magnetic resonance signal and a diffuse magnetic resonance signal; and
taking the structural-state magnetic resonance signal and the diffuse magnetic resonance signal as the second feature.

In some embodiments, the obtaining the semantic map according to the fitting result and the preset brain map further comprises:
mapping the fitting result to the preset brain map, and decoding a mapped fitting result by a self-encoder; and
selecting semantic information in each target point that generates the strongest magnetic resonance representation respectively according to a decoding result, and generating the semantic map.

In some embodiments, the generating the cognitive training material according to the target semantic information and the weight parameter of the deep learning model further includes:
acquiring a shallow weight parameter and a deep weight parameter of the deep learning model; and
generating the cognitive training material according to the target semantic information as input information, the shallow weight parameter, and the deep weight parameter.

In a second aspect, a cognitive training method is further provided in the present disclosure, including:
acquiring a target point for which a user needs to perform cognitive training, and determining semantic information corresponding to the target point based on a semantic map;
acquiring a cognitive training material according to the semantic information, the cognitive training material being obtained based on the cognitive training material generation method in the above first aspect; and
presenting the cognitive training material to the user in accordance with a preset duration.

In a third aspect, an electronic device is further provided in the present disclosure, including a memory, a processor, and a computer program stored on the memory and capable of running on the processor. The processor is configured to execute the computer program to perform the cognitive training material generation method in the above first aspect.

In a fourth aspect, a computer-readable storage medium is further provided in the present disclosure, storing a computer program. The computer program is executed by a processor to implement the cognitive training material generation method in the above first aspect.

Details of one or more embodiments of the present disclosure are set forth in the following accompanying drawings and descriptions. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in the embodiments of the present application or the related technology, the accompanying drawings to be used in the description of the embodiments or the related technology will be briefly introduced below, and it will be obvious that the accompanying drawings in the following description are only some of the embodiments of the present application, and that, for one skilled in the art, other accompanying drawings can be obtained based on these accompanying drawings without putting in creative labor.

FIG. 1 is a flowchart of a cognitive training material generation method in an embodiment of the present disclosure.

FIG. 2 is a flowchart of a cognitive training method in an embodiment of the present disclosure.

FIG. 3 is a flowchart of a cognitive training method based on magnetic resonance and graph convolution in an embodiment of the present disclosure.

FIG. 4 is a schematic diagram of an experimental design paradigm of a consecutive identification task in an embodiment of the present disclosure.

FIG. 5 is a schematic diagram of a cognitive training material in an embodiment of the present disclosure.

### DETAILED DESCRIPTION OF THE EMBODIMENT

The technical solutions in the embodiments of the present disclosure will be described clearly and completely in the following in conjunction with the accompanying drawings in the embodiments of the present disclosure, and it is obvious that the described embodiments are only a part of the embodiments of the present disclosure, but not all of the embodiments. Based on the embodiments in the present disclosure, all other embodiments obtained by one skilled in the art without making creative labor fall within the scope of protection of the present disclosure.

Unless defined otherwise, technical terms or scientific terms involved in the present disclosure have the same meanings as would generally understood by one skilled in the technical field of the present disclosure. In the present disclosure, “a”, “an”, “one”, “the”, and other similar words do not indicate a quantitative limitation, which may be singular or plural. The terms such as “comprise”, “include”, “have”, and any variants thereof involved in the present disclosure are intended to cover a non-exclusive inclusion. For example, processes, methods, systems, products, or devices including a series of steps or modules (units) are not limited to these steps or modules (units) listed, and may include other steps or modules (units) not listed, or may include other steps or modules (units) inherent to these processes, methods, systems, products, or devices. Words such as “join”, “connect”, “couple”, and the like involved in the present disclosure are not limited to physical or mechanical connections, and may include electrical connections, whether direct or indirect. “A plurality of” involved in the present disclosure means two or more. The term “and/or” describes an association relationship between associated objects and represents that three relationships may exist. For example, A and/or B may represent the following three cases: only A exists, both A and B exist, and only B exists. Generally, a character “/” indicates an “or” relationship between the associated objects. The terms “first”, “second”, “third”, and the like involved in the present disclosure are only intended to distinguish similar objects and do not represent specific ordering of the objects.

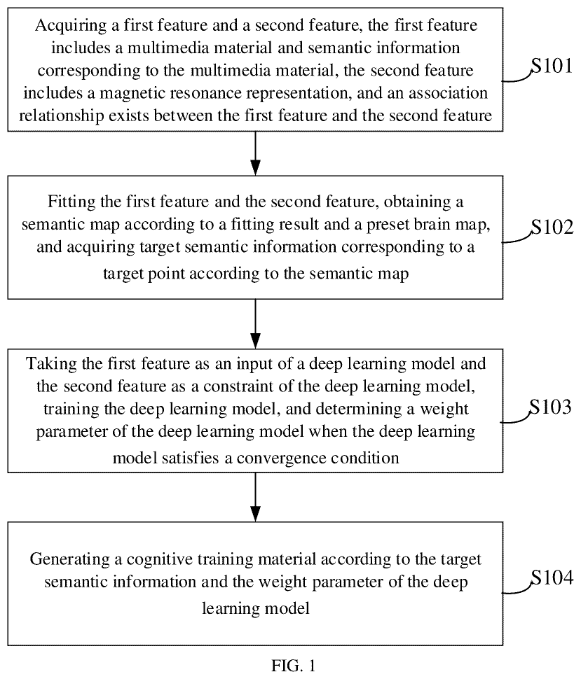

A cognitive training material generation method is provided in the present embodiment, and FIG. 1 is a flowchart of the cognitive training material generation method of the present embodiment. Referring to FIG. 1, the cognitive training material generation method includes following step 101 to step 104.

Step 101 includes acquiring a first feature and a second feature, the first feature includes a multimedia material and semantic information corresponding to the multimedia material, the second feature includes a magnetic resonance representation, and an association relationship exists between the first feature and the second feature.

The multimedia material may include natural stimuli such as graphs, videos, sounds, and other natural stimuli that will produce visual, auditory, and other stimuli to a subject, and the semantic information of the multimedia material may include a textual description of the natural stimuli. Alternatively, a feature of the multimedia material and a feature of the semantic information of the multimedia material may be taken as the first feature.

The association relationship between the first feature and the second feature may include: providing the first feature to the subject, and generating the second feature when the subject recognizes the first feature. The acquiring the second feature may include: acquiring a magnetic resonance signal generated by the subject during recognition of the natural stimuli. Alternatively, the subject may recognize a series of the natural stimuli, including visual and auditory stimuli, in a magnetic resonance machine and determine whether each stimulus has appeared before by pressing a key, and a magnetic resonance signal generated by the subject in the experiment may be acquired. A feature of the magnetic resonance signal may be extracted to obtain a magnetic resonance representation.

Step 102 includes fitting the first feature and the second feature, obtaining a semantic map according to a fitting result and a preset brain map, and acquiring target semantic information corresponding to a target point according to the semantic map.

The multimedia material and the semantic information corresponding to the multimedia material and the magnetic resonance representation may be fitted, and a fitted weight may be obtained after the fitting. The fitted weight may be corresponded to the preset brain map to obtain a relationship between different categories of semantic information and responses of various points in a cerebral cortex, and a semantic map may be generated. A target semantic category corresponding to the target point may be acquired according to the semantic map, and target semantic information related to the target semantic category may be generated.

Step 103 includes taking the first feature as an input of a deep learning model and the second feature as a constraint of the deep learning model, training the deep learning model, and determining a weight parameter of the deep learning model when the deep learning model satisfies a convergence condition.

Training the deep learning model may include taking a portion of the first feature and corresponding second feature as a training set, taking the remaining first feature and the remaining second feature as a test set, comparing similarity between an output of the deep learning model and the second feature, and determining whether the deep learning model satisfies the convergence condition. A trained deep learning model is capable of predicting and outputting a magnetic resonance representation generated by the subject in recognizing the multimedia material and the semantic information corresponding to the multimedia material based on the multimedia material and the semantic information corresponding to the multimedia material.

Step 104 includes generating a cognitive training material according to the target semantic information and the weight parameter of the deep learning model.

The target semantic information may be obtained by first acquiring a target semantic information category, and then acquiring a relevant description of the target semantic information category. The target semantic information may be input into a neural network generation model, the weight parameter of the deep learning model may be taken as a constraint of the neural network generation model, and the cognitive training material related to the target semantic information may be output by the neural network generation model. Exemplarily, the weight parameter of the deep learning model may include a shallow weight parameter and a deep weighting parameter, the shallow weighting parameter is configured to constrain detail information of the generated material better, and the deep weight parameter is configured to constrain the semantic information of the generated material better.

By the above steps, the second feature including responses of target points in the cerebral cortex may be obtained according to the first feature including the multimedia material and the corresponding semantic information, the multimedia material may provide auditory and visual multifaceted stimuli, which better stimulates activity of the brain, so that the semantic map established by the first feature and the second feature may be more accurate, and it is possible to accurately determine corresponding semantic information according to the target point in the cerebral cortex by the semantic map. The deep learning model may be trained according to the first feature and the second feature, so that the deep learning model may learn a relationship between a real response of the target point in the cerebral cortex and the cognitive training material. In the process of generating the cognitive training material according to the semantic information, weight data of the deep learning model may be taken as a condition to constrain the detailed information and semantic information of the cognitive training material better, so that the generated cognitive training material can accurately and efficiently stimulate a brain region where the target point in the cerebral cortex is located, thereby solving a problem of not being able to acquire the cognitive training material that can accurately stimulate the functional cortical target points in the brain that need to be trained, and realizing the acquiring of the cognitive training material based on an individual subject and maximizing the training of the brain of the subject.

In some embodiments, the generating the cognitive training material may further include: inputting a first cognitive training material generated according to the target semantic information and the weight parameter of the deep learning model into the deep learning model, and predicting a second feature corresponding to the first cognitive training material; determining whether the second feature corresponding to the first cognitive training material satisfies a preset condition; and screening the first cognitive training material according to a determining result to obtain a second cognitive training material.

The inputting the first cognitive training material into the deep learning model may further include: acquiring the first cognitive training material and corresponding semantic information, extracting a feature of the first cognitive training material, encoding the semantic information, and inputting the feature of the first cognitive training material and encoded semantic information to the deep learning model. The deep learning model may predict a response generated by the target point according to the first cognitive training material, and obtain a second feature corresponding to the first cognitive training material. It is determined whether the second feature corresponding to the first cognitive training material satisfies the preset condition to screen the first cognitive training material.

Exemplarily, the deep learning model may be a fusion model including a graph model and a data processing model, which are configured to establish a mapping relationship among the multimedia material and a corresponding semantic description of the multimedia material and the magnetic resonance representation. Alternatively, the graph model may include a GCN (Graph Convolutional Network), a GAT (Graph Attention Network), a GraphSage (Graph Sample and Aggregate), or other models, which may enable the semantic information and the magnetic resonance representation to match better and the semantic information to be represented better. According to a data modality of the multimedia material, a corresponding data processing model may be fused based on the graph model. Alternatively, for graph data, the data processing model may include a resnet (Residual Network), a Vit (Vision transformer), a mask-RCNN (Mask Region-based Convolutional Neural Network), or other models. Data processing of the multimedia material may be achieved by the data processing model.

In some embodiments, the acquiring the first feature further includes: extracting a feature of the multimedia material by a convolutional neural network, encoding semantic information of the multimedia material, and taking the feature of the multimedia material and encoded semantic information as the first feature.

Alternatively, the features of the multimedia material may be obtained by feature extraction of the multimedia material via a residual convolutional network. The multimedia material may include graphs, videos, sounds, and other natural stimuli that will produce visual, auditory, and other stimuli to the subject, and include a plurality of modalities. The semantic information of the multimedia material may be a textual modality of the natural stimuli. The semantic information of the multimedia material may be encoded by an encoding part of a self-encoder to obtain a feature of the semantic information of the multimedia material. According to corresponding modal data, coding methods such as one-hot code, graph embedding, audio embedding, and word embedding may be selected. Features of the encoded semantic information may be combined by concatenation, point multiplication, or cross-attention algorithms. The data obtained after combining may be linearly or non-linearly fitted to the magnetic resonance signal.

The acquiring the second feature may further include: acquiring a functional magnetic resonance signal corresponding to the first feature, and extracting a feature of the functional magnetic resonance signal, and obtaining a signal feature; and mapping the signal feature to a cerebral cortex, and taking a mapped signal feature as the second feature. After taking the mapped signal feature as the second feature, the method may further include: acquiring a structural-state magnetic resonance signal and a diffuse magnetic resonance signal; taking the structural-state magnetic resonance signal and the diffuse magnetic resonance signal as the second feature.

The functional magnetic resonance signal may be a magnetic resonance signal acquired during functional magnetic resonance imaging, the structural-state magnetic resonance signal may be a structural-state magnetic resonance signal acquired during structural-state magnetic resonance imaging, and the diffuse magnetic resonance signal is a magnetic resonance signal acquired during magnetic resonance diffusion imaging. Alternatively, the feature of the functional magnetic resonance signal may be extracted by a generalized linear model, and a standard template of a cortical surface may be selected to map the feature of the functional magnetic resonance signal to the cerebral cortex.

The obtaining the semantic map according to the fitting result and the preset brain map may further include: mapping the fitting result to the preset brain map, and decoding a mapped fitting result by a self-encoder; and selecting semantic information in each target point that generates the strongest magnetic resonance representation respectively according to a decoding result, and generating the semantic map. The decoding the mapped fitting result by the self-encoder may further include: when generating the semantic map, decoding a feature of a vertex by a decoding portion of the self-coder. Alternatively, before and after decoding by the self-encoder, principal component analysis, a Topk algorithm and the like may be selected for noise reduction.

The generating the cognitive training material according to the target semantic information and the weight parameter of the deep learning model may further include: acquiring a shallow weight parameter and a deep weight parameter of the deep learning model; and generating the cognitive training material according to the target semantic information as input information, the shallow weight parameter, and the deep weight parameter. Alternatively, the cognitive training material may be generated by a neural network generation model. The neural network generation model may be a fusion model including a diffusion model. The shallow weight parameter and the deep weight parameter may be input into the neural network generation model as the constraint, the shallow weight parameter is configured to constrain the detail information of the generated material better, and the deep weight parameter is configured to constrain the semantic information of the generated material better.

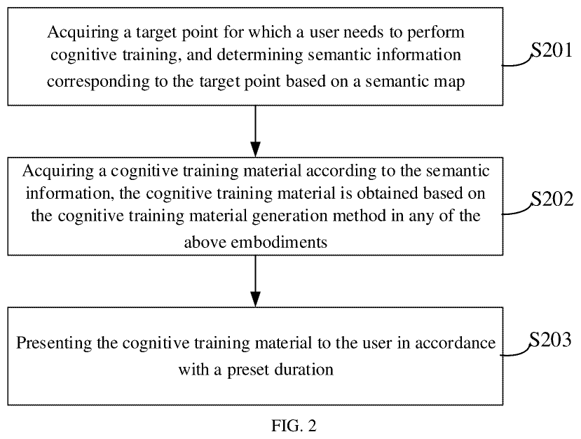

In some embodiments, a cognitive training method is provided, and FIG. 2 is a flowchart of the cognitive training method in the present embodiment. The cognitive training method includes step 201 to step 203.

Step 201 includes acquiring a target point for which a user needs to perform cognitive training, and determining semantic information corresponding to the target point based on a semantic map.

Step 202 includes acquiring a cognitive training material according to the semantic information, the cognitive training material is obtained based on the cognitive training material generation method in any of the above embodiments.

Step 203 includes presenting the cognitive training material to the user in accordance with a preset duration.

Alternatively, half an hour may be spent every day to show the cognitive training material to the user, and the user may recognize the cognitive training material and stimulate the relevant brain region corresponding to the target point, so as to achieve effect of enhancing cognition of the user.

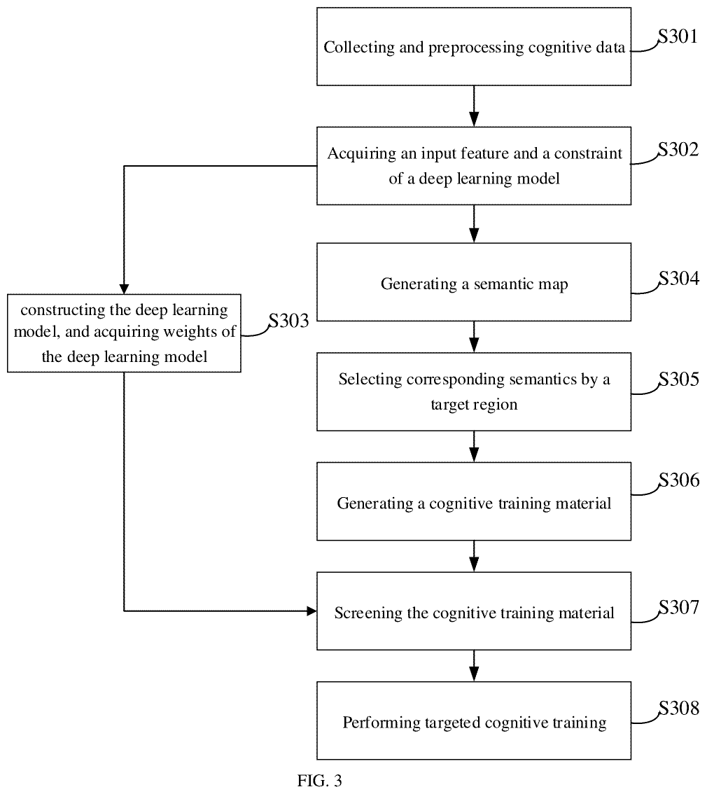

In the present embodiment, a cognitive training method based on magnetic resonance and graph convolution is further provided. FIG. 3 is a flowchart of the cognitive training method based on magnetic resonance and graph convolution in the present embodiment. Referring to FIG. 3, the flowchart may include step 301 to step 308.

Step 301 may include collecting and preprocessing cognitive data. Taking graph-based data collection and preprocessing as an example, a coco (Common Objects in Context) graph data set may be acquired, and 10,000 natural graphs and labels and description information corresponding to the natural graphs may be randomly obtained from the graph data set. Perform consecutive identification of large-scale color natural graphs for the subject that meets experimental requirements, and task-state functional magnetic resonance signals during identification may be collected. Alternatively, the subject that meets the experimental requirements may be: healthy subjects aged from 18 to 30 years old, with normal or corrected normal vision, all right-handed, without serious health problems, cognitive or psychiatric disorders, such as stroke, epilepsy, heart disease, etc., and without implantation of metal objects or pacemakers in the body, etc., and most importantly, the subjects are able to comprehend experimental process and requirements, and are capable of cooperating in completing experimental tasks.

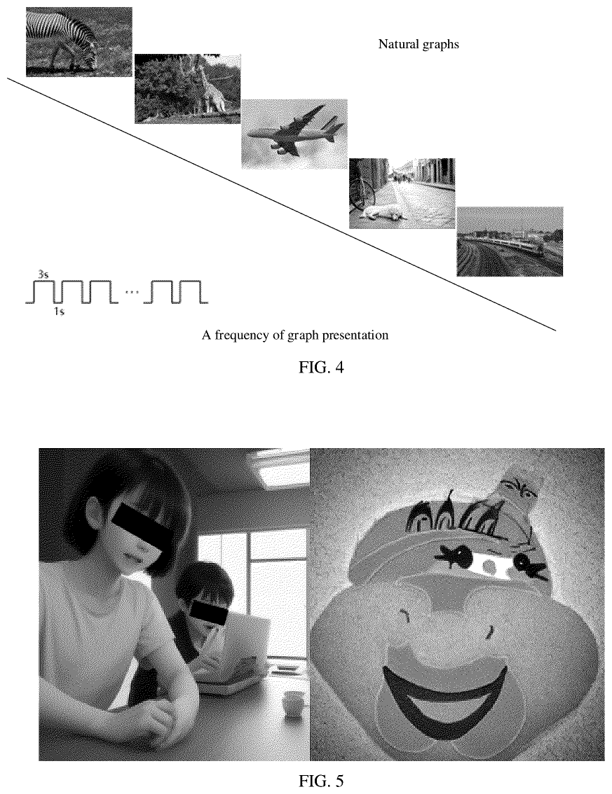

FIG. 4 is a schematic diagram of an experimental design paradigm of a consecutive identification task. Referring to FIG. 4, in the experiment process, the subjects may lie in a magnetic resonance machine, view the natural graphs, and simultaneously perform graph recognition. Each subject may accept a total of 40 magnetic resonance scans, during which the subject may be required to focus on viewing the graphs, recognize the graphs, and press a key when the graphs have appeared before. 10,000 different graphs may appear 3 times randomly, with a uniformly distributed probability of appearance, and an order of the graphs may be fixed between the subjects. 90% of the graphs viewed by each subject may be different from that viewed by the other subjects and 10% of the graphs viewed by each subject may be shared with the other subjects. In an experiment, the subjects may perform a total of 12 runs, each with 75 trials, of which the first 3 and last 4 trials have no graphs displayed and the subjects may see a blank interface, and the remaining 68 trials may be divided into 63 stimulus trials and 5 blank trials, which were randomly placed in the runs. Even numbered runs may have 62 stimulus trials and a 63rd blank trial. Therefore, a total of 750 graph stimuli may be presented in a single task of viewing of graphs and consecutive recognition. Alternatively, a frequency of graph presentation may be 3 seconds per graph presentation and 1 second between graph presentations.

Step 302 may include acquiring an input feature and a constraint of a deep learning model. The natural graphs may be cropped and features of the natural graphs may be extracted by a residual convolution network. A size of the cropped graphs may be (425, 425, 3), 425 may correspond to a length or a width of the graphs, and 3 may correspond to red, yellow, and green color channels. To encode the semantic information of the graphs, models including coding and decoding may be used such as a UNet model, a Transformer model, and one-hot code. Taking coding by one-hot code as an example, the semantic information of the graphs may be coded by one-hot code to get multihots. Specifically, each natural graph may include data of 80 dimensions, 80 refers to the number of categories of specific things in the coco graph set. When the graph includes a category, a value of the multihots in this category may be set to 1, otherwise the value of the multihots in this category may be set to 0. Because each graph may include a plurality of categories, multihots may include a plurality of 1. Extracted graph features and multihots may be taken together as the input feature of the deep learning model.

A response signal may be acquired from the subject when the subject views a set of the graphs, and the response signal may be the task-state functional magnetic resonance signal. The feature of the task-state functional magnetic resonance signal may be extracted by a generalized linear model to obtain a beta value, and the beta value may represent an activation level of the brain region of the subject. At this point, the extracted beta value may be a voxel-based feature. A standard fsaverage template (the standard template of the cortical surface) may be selected to map the beta value to the cerebral cortex to obtain a beta feature. The beta value may be mapped to the cerebral cortex to obtain the beta feature, and the mapped beta feature may be taken as the constraint for the deep learning model.

A portion of the input feature and corresponding constraint of the deep learning model may be taken as a training set, and the remaining input feature and the remaining constraint of the deep learning model may be taken as a test set. The training set may include the graph features and response features corresponding to different 9000 graphs viewed by each subject, and the test set may include the graph features and response features corresponding to 1000 graphs viewed by all subjects together.

Step 303 may include constructing the deep learning model. Exemplarily, the deep learning model may be a fusion model including a graph model. The fusion model including the graph model may be constructed based on a Vision GNN (ViG, vision-based graph neural network) model. An MLP (Multi-Layer Perceptron) layer may be added to an output of the ViG model, which ensures that a shape of output data is consistent with a shape of the beta feature, so that the fusion model including the graph model may predict corresponding second feature according to the first feature of the input. fsaverage7 and fsaverage5 belong to a set of standard templates including different resolutions provided by open-source magnetic resonance data processing software named FreeSurfer, and the beta value of the model input may be unified to the resolution of fsaverage7. Since a limitation of a video memory size, the resolution 327684 (covering left and right hemibrain) of fsaverage7 (high-resolution fsaverage7 grid) may be down sampled to fsaverage5, so that the resolution may be converted to the resolution of fsaverage5 20484 (covering left and right hemibrain). A bert model may be selected to process the semantic information of the graphs. An MLP layer may be added to an output of the bert model as well to ensure that a shape of output data is consistent with the shape of the beta feature. Alternatively, the graph model may be selected as a model such as a resnet, a mask-RCNN (Region-Convolutional Neural Network), a diffusion model, etc.

A structural-state magnetic resonance signal and a diffuse magnetic resonance signal may be acquired. The structural-state magnetic resonance signal and the diffuse magnetic resonance signal may be processed by the graph convolution model, respectively, to obtain a structural-state magnetic resonance feature and a diffuse magnetic resonance feature. The structural-state magnetic resonance feature and the diffuse magnetic resonance feature may be in a shape of (20484,), i.e., uniformly at the resolution of fsaverage5. Alternatively, when constructing the fusion model including the graph model, it may be selected according to actual situation whether to introduce the structural-state magnetic resonance feature and the diffuse magnetic resonance feature as a priori information of the model.

A graph feature output from the graph model, a feature of the semantic information of the graph output from the bert model, and processed structural-state magnetic resonance feature and diffuse magnetic resonance feature may be concatenated to obtain data with a shape of (20484, 4). Alternatively, a cross-attention mechanism may be used to realize the fusion of multiple features. The concatenated data may be processed by a convolutional layer to output data with a shape of (20484,). The obtained data may be normalized to (−1, 1) by a softmax layer to acquire a similarity coefficient of a predicted value and the beta feature. The similarity coefficient is configured to determine effectiveness of the prediction of the deep learning model and train the model to convergence. A loss function of the fusion model including the graph model may be as follows:

$$Loss = 1 - Corr ( {Pred}_{softmax} , β )$$

The Corr() function is configured to acquire the similarity coefficient, Pred_{softmax} may represent the predicted value of the output of the fusion model including the graph model, β is the beta feature, and an Adam optimizer may be selected as a model optimizer.

Hyperparameters such as a model learning rate, the number of graph convolutional layers, the number of convolutional layers, a convolutional kernel parameter, the number of channels, and the like of the fusion model including the graph model may be optimized by methods such as a grid search method and a random search method, and in conjunction with cross-validation. Knowledge distillation may further be performed on the fusion model including the graph model. Alternatively, the semantic information of the graph may be introduced as priori information in the distillation process, and the distilled fusion model including the graph model may reduce the number of weight parameters of the fusion model to achieve effect of improving prediction accuracy of the fusion model.

Step 304 may include generating a semantic map. The multihots and beta values mapped to the cortex may be fitted, and fitted weights may be corresponded to a standard brain map. Alternatively, the standard brain map may be a Brainnetome Atlas. The fitted weights corresponding to the standard brain map are decoded, and the decoding process may be essentially a process of finding a relationship between semantics of the graph and the response of the brain cortex. Alternatively, a plurality of semantic categories may be generated, each of which may include a plurality of specific words, each of which has a different color, and the words with different colors correspond to the cerebral cortex responses of different brain regions. After decoding, the strongest expressed semantic category may be selected to obtain the semantic map.

Step 305 may include selecting corresponding semantics by a target region. A target for cognitive training may be selected from the semantic map according to requirements. For example, a fusiform face area (FFA) may be selected, the semantic information category corresponding to the fusiform face area may be obtained according to the semantic map as “human”, and a description associated with the category, i.e., a description associated with “human” may be obtained, such as “happy person” and “college student”.

Step 306 may include generating a cognitive training material. The description such as “happy person” and “college student” associated with the semantic category may be input into the neural network generation model to generate a large number of semantically related graph materials. FIG. 5 is a schematic diagram of the cognitive training material in the present embodiment. Alternatively, the neural network generation model may be a stable diffusion model or the fusion model including the diffusion model. The neural network generation model is configured to generate the cognitive training material, and the shallow weight parameter and the deep weight parameter of the deep learning model may be introduced to an input of the neural network generation model as constraint information, in addition to semantic information corresponding to the target point input to the neural network generation model.

Step 307 may include screening the cognitive training material. According to the method in step 302, feature extraction may be performed on the cognitive training material or other image material for training the deep learning model. The weights of the deep learning model constructed in step 303 may be acquired. According to the extracted feature and the weight of the trained deep learning model, a target point response corresponding to the extracted feature may be predicted. The material may be further screened according to the predicted the response signal of the FFA target point corresponding to the material. Training the neural network generation model may include: adjusting the neural network generation model according to a prediction result of the deep learning model, or fixing the feature extraction and the weight of the trained deep learning model, directly accessing the neural network generation model, and further optimizing the neural network generation model.

Step 308 may include performing targeted cognitive training. The screened cognitive training material is configured for cognitive training, and the screened cognitive training material may drive peak activity in a target brain region beyond a naturally occurring level.

The cognitive training process may include: with a goal of improving attention and memory of an individual subject, selecting parietal and frontal target points on a semantic map, and generating and screening graph material by the above neural network generation model. Two groups of subjects may be recruited to conduct a training experiment and a contrast experiment. First the training group carried out a short-term graph memory experiment, in which the training material generated at the previous step is organized, collected, and disordered to serve as the stimuli for this experiment. This experiment may be completed within a MRI device, during which eight stimuli may be randomly selected from stimulus material and presented to the subjects in sequence, pushing the peak activity of the target brain region beyond the naturally occurring level. Each stimulus may appear at a fixed time, then two of the stimuli may be randomly deleted and the other six stimuli may be played back, the subjects may be allowed to recall which stimuli do not appear and provide feedback to a main subject in a form of a sound or a finger key press after completion of stimuli playback. An experimental paradigm and a training paradigm of a contrast group may be similar to those of a training group, with difference that the stimuli presented to the subjects in the contrast experiment are natural graphs that have not been trained by graph convolution. After displaying the graph material sequentially, the subjects need to be asked to recognize which stimuli do not appear after replaying the stimuli.

Magnetic resonance data may be collected from both groups of the subjects and behavioral data may be recorded. It may be determined whether experimental stimuli in the are more conducive to training concentration in the experimental group by accuracy contract. An activation result of the magnetic resonance data may also serve as key evidence of effectiveness of the training experimental material. In other words, the magnetic resonance data may be preprocessed and analyzed with a general linear model, and a statistical test at a group level may be performed to obtain that the brain regions with stronger activation in the experimental group than in the contrast group are located at the target points of the cognitive training, thereby further proving the effectiveness of the experimental material.

Finally, the subjects may be shown the screened cognitive training material in accordance with a preset duration. The subjects may view the training material for half an hour every day to stimulate the brain regions of the subjects related to attention and memory.

By the above steps, a consecutive recognition task based on natural stimuli may be carried out, and stimulus semantic information may be collected and magnetic resonance signals may be acquired. The natural stimuli and stimulus semantic information may be encoded as input of the deep learning model, and features may be extracted from the magnetic resonance signals as the constraint of the deep learning model, and the constructed deep learning model may be trained to obtain weight parameters of the trained model. The input and the constraint of the model may be fitted, and the fitted weights may be corresponded to the standard brain map and decoded to obtain the semantic map. The required target points may be selected according to the semantic map, and the semantic information corresponding to the target points may be input into the constructed neural network generation model to generate cognitive training material. The cognitive training material may be subjected to feature extraction and model prediction, and the cognitive training material may be further screened according to target point response. The screened cognitive training material is configured for the cognitive training of the subjects. The filtered cognitive training material is used for cognitive training of the subjects. The present method may enable non-invasive and measurable cognitive training of the cortical target points by magnetic resonance and graph convolution.

It is noted that the steps illustrated in the above-described process or in the flowchart of the accompanying drawings may be performed in a computer system, such as a set of computer-executable instructions, and that, although a logical sequence is illustrated in the flowchart, in some instances the steps illustrated or described may be performed in a different order from that shown herein.

An electronic device is further provided in an embodiment, including a memory, a processor, and a computer program stored on the memory and capable of running on the processor. The processor is configured to execute the computer program to perform the steps of methods in any one of the above embodiments.

Alternatively, the electronic device may further include a transmission device and an input/output device. The transmission device may be connected to the processor and the input/output device may be connected to the processor.

Alternatively, in the present embodiment, the processor is configured to perform the following steps via the computer program.

Step 101 includes acquiring a first feature and a second feature, the first feature includes a multimedia material and semantic information corresponding to the multimedia material, the second feature includes a magnetic resonance representation, and an association relationship exists between the first feature and the second feature.

Step 102 includes fitting the first feature and the second feature, obtaining a semantic map according to a fitting result and a preset brain map, and acquiring target semantic information corresponding to a target point according to the semantic map.

Step 103 includes taking the first feature as an input of a deep learning model and the second feature as a constraint of the deep learning model, training the deep learning model, and determining a weight parameter of the deep learning model when the deep learning model satisfies a convergence condition.

Step 104 includes generating a cognitive training material according to the target semantic information and the weight parameter of the deep learning model.

It is noted that specific examples in this embodiment may refer to the examples described in the above embodiments and alternative implementations, and will not be repeated in the present embodiment.

In addition, in conjunction with the cognitive training material generation method provided in the above embodiments, a storage medium may also be provided in the present embodiment for implementation. A computer program is stored in the storage medium, and the computer program is executed by a processor to implement any of the cognitive training material generation methods of the above embodiments.

The specific embodiments described herein are only used to explain this application and are not intended to qualify it. According to the embodiments provided in the present disclosure, all other embodiments obtained by one skilled in the art without creative labor are within the scope of protection of the present disclosure.

Obviously, the accompanying drawings are only some examples or embodiments of the present disclosure, and it is also possible for one skilled in the art to apply the present application to other similar situations in accordance with these accompanying drawings without creative labor. Furthermore, it is to be understood that although the work done in this development process may be complex and lengthy, certain changes in design, manufacturing, or production, etc., based on the technical content disclosed in the present disclosure are only conventional technical means to one skilled in the art and should not be considered insufficient for the disclosure of the present disclosure.

The term “embodiment” as used in the present disclosure, means that specific features, structures, or characteristics described in conjunction with embodiments may be included in at least one embodiment of the present disclosure. The appearance of the phrase at various locations in the specification does not necessarily imply the same embodiment, nor does it imply independence or optionality from other embodiments that are mutually exclusive. It will be clearly or implicitly understood by one skilled in the art that the embodiments described in the present disclosure may be combined with other embodiments without conflict.

The above-described embodiments express only several embodiments of the present application, which are described in a more specific and detailed manner, but are not to be construed as a limitation on the scope of patent protection. For one skilled in the art, several deformations and improvements can be made without departing from the conception of the present application, which all fall within the scope of protection of the present application. Therefore, the scope of protection of this application shall be subject to the attached claims.

What is claimed is:

1. A cognitive training material generation method, comprising:
acquiring a first feature and a second feature, wherein the first feature comprises a multimedia material and semantic information corresponding to the multimedia material, the second feature comprises a magnetic resonance characterization, and an association relationship exists between the first feature and the second feature;
fitting the first feature and the second feature, obtaining a semantic map according to a fitting result and a preset brain map, and acquiring target semantic information corresponding to a target point according to the semantic map;
taking the first feature as an input of a deep learning model and the second feature as a constraint of the deep learning model, training the deep learning model, and determining a weight parameter of the deep learning model when the deep learning model satisfies a convergence condition; and
generating a cognitive training material according to the target semantic information and the weight parameter of the deep learning model.

2. The cognitive training material generation method of claim 1, wherein the generating the cognitive training material further comprises:
inputting a first cognitive training material generated according to the target semantic information and the weight parameter of the deep learning model into the deep learning model, and predicting a second feature corresponding to the first cognitive training material;
determining whether the second feature corresponding to the first cognitive training material satisfies a preset condition; and
screening the first cognitive training material according to a determining result to obtain a second cognitive training material.

3. The cognitive training material generation method of claim 1, wherein the acquiring the first feature further comprises:
extracting a feature of the multimedia material by a convolutional neural network, encoding the semantic information of the multimedia material, and taking the feature of the multimedia material and encoded semantic information as the first feature.

4. The cognitive training material generation method of claim 1, wherein the acquiring the second feature further comprises:
acquiring a functional magnetic resonance signal corresponding to the first feature, and extracting a feature of the functional magnetic resonance signal, and obtaining a signal feature; and
mapping the signal feature to a cerebral cortex, and taking a mapped signal feature as the second feature.

5. The cognitive training material generation method of claim 4, wherein after taking the mapped signal feature as the second feature, the method further comprises:
acquiring a structural-state magnetic resonance signal and a diffuse magnetic resonance signal; and
taking the structural-state magnetic resonance signal and the diffuse magnetic resonance signal as the second feature.

6. The cognitive training material generation method of claim 1, wherein the obtaining the semantic map according to the fitting result and the preset brain map further comprises:
mapping the fitting result to the preset brain map, and decoding a mapped fitting result by a self-encoder; and
selecting semantic information in each target point that generates the strongest magnetic resonance representation respectively according to a decoding result, and generating the semantic map.

7. The cognitive training material generation method of claim 1, wherein the generating the cognitive training material according to the target semantic information and the weight parameter of the deep learning model further comprises:
acquiring a shallow weight parameter and a deep weight parameter of the deep learning model; and
generating the cognitive training material according to the target semantic information as input information, the shallow weight parameter, and the deep weight parameter.

8. A cognitive training method, comprising:
acquiring a target point for which a user needs to perform cognitive training, and determining semantic information corresponding to the target point based on a semantic map;
acquiring a cognitive training material according to the semantic information, wherein the cognitive training material is obtained based on the cognitive training material generation method of claim 1; and
presenting the cognitive training material to the user in accordance with a preset duration.

9. An electronic device, comprising a memory and a processor, wherein a computer program is stored in the memory, and the processor is configured to execute the computer program to perform the cognitive training material generation method of claim 1.

10. The electronic device of claim 9, wherein the generating the cognitive training material further comprises:
inputting a first cognitive training material generated according to the target semantic information and the weight parameter of the deep learning model into the deep learning model, and predicting a second feature corresponding to the first cognitive training material;
determining whether the second feature corresponding to the first cognitive training material satisfies a preset condition; and
screening the first cognitive training material according to a determining result to obtain a second cognitive training material.

11. The electronic device of claim 9, wherein the acquiring the first feature further comprises:
extracting a feature of the multimedia material by a convolutional neural network, and encoding semantic information of the multimedia material, and taking the feature of the multimedia material and encoded semantic information as the first feature.

12. The electronic device of claim 9, wherein the acquiring the second feature further comprises:
acquiring a functional magnetic resonance signal corresponding to the first feature, and extracting a feature of the functional magnetic resonance signal, and obtaining a signal feature; and
mapping the signal feature to a cerebral cortex, and taking a mapped signal feature as the second feature.

13. The electronic device of claim 12, wherein after taking the mapped signal feature as the second feature, the method further comprises:
acquiring a structural-state magnetic resonance signal and a diffuse magnetic resonance signal; and
taking the structural-state magnetic resonance signal and the diffuse magnetic resonance signal as the second feature.

14. The electronic device of claim 9, wherein the obtaining the semantic map according to the fitting result and the preset brain map further comprises:
mapping the fitting result to the preset brain map, and decoding a mapped fitting result by a self-encoder; and
selecting semantic information in each target point that generates the strongest magnetic resonance representation respectively according to a decoding result, and generating the semantic map.

15. The electronic device of claim 9, wherein the generating the cognitive training material according to the target semantic information and the weight parameter of the deep learning model further comprises:
acquiring a shallow weight parameter and a deep weight parameter of the deep learning model; and
generating the cognitive training material according to the target semantic information as input information, the shallow weight parameter, and the deep weight parameter.

16. A computer-readable storage medium, storing a computer program, wherein the computer program is executed by a processor to implement steps of the cognitive training material generation method of claim 1.

17. The computer-readable storage medium of claim 16, wherein the generating the cognitive training material further comprises:
inputting a first cognitive training material generated according to the target semantic information and the weight parameter of the deep learning model into the deep learning model, and predicting a second feature corresponding to the first cognitive training material;
determining whether the second feature corresponding to the first cognitive training material satisfies a preset condition; and
screening the first cognitive training material according to a determining result to obtain a second cognitive training material.

18. The computer-readable storage medium of claim 16, wherein the acquiring the first feature further comprises:
extracting a feature of the multimedia material by a convolutional neural network, and encoding semantic information of the multimedia material, and taking the feature of the multimedia material and encoded semantic information as the first feature.

19. The computer-readable storage medium of claim 16, wherein the acquiring the second feature further comprises:
acquiring a functional magnetic resonance signal corresponding to the first feature, and extracting a feature of the functional magnetic resonance signal, and obtaining a signal feature; and
mapping the signal feature to a cerebral cortex, and taking a mapped signal feature as the second feature.

20. The computer-readable storage medium of claim 19, wherein after taking the mapped signal feature as the second feature, the method further comprises:
acquiring a structural-state magnetic resonance signal and a diffuse magnetic resonance signal; and
taking the structural-state magnetic resonance signal and the diffuse magnetic resonance signal as the second feature.
